(19) United States
(12) Patent Application Publication
(10) Pub. No.: US 2023/0211337 A1
(43) Pub. Date: Jul. 6, 2023

(54) CONTAINER SYSTEM FOR RECEIVING A LIQUID SAMPLE

(71) Applicants: GESVAL S.A., Angleur (BE); UNIVERSITÉ DE LIÈGE, Liege (BE)

(72) Inventors: Fabrice BUREAU, Ransart (BE); Clément BUREAU, Ransart (BE); Laurent GILLET, Tenneville (BE); Michael VERLEYEN, Liège (BE)

(21) Appl. No.: 18/016,194

(22) PCT Filed: Jul. 13, 2021
(86) PCT No.: PCT/EP2021/069494

§ 371 Date: Jan. 13, 2023

(30) Foreign Application Priority Data
Jul. 15, 2020 (EP) 20186086.3
Oct. 27, 2020 (EP) 20204123.2

(51) Int. Cl. B01L 3/00

(52) U.S. Cl. CPC B01L 3/502; B01L 2300/0832; B01L 2200/0605; B01L 2300/042; B01L 2400/0605

(57) ABSTRACT

A container system for receiving a liquid sample has a receptacle including a substantially tubular portion for receiving a predetermined volume of the sample. The substantially tubular portion has a first open end and an at least partially openable second end; and a chamber at least partially surrounding the substantially tubular portion. The chamber has an at least partially open upper portion and a closed bottom.

### TECHNICAL FIELD OF THE INVENTION

The present invention relates to the field of container systems for receiving a liquid sample.

### PRIOR ART

The current pandemic generated by the SARS-CoV-2 virus revealed the need to carry out diagnostic tests in a relatively rapid and generalised manner. It has been found indispensable to be able to test a large number of persons in very short times with a view to taking the necessary precautionary measures to prevent rapid and exponential propagation of the disease. The diagnostic tests used at present to detect the SARS-CoV-2 virus usually necessitate taking a nose and throat sample by means of a swab. This is a constraint because it necessitates qualified personnel to carry it out.

A sample that is relatively easy to take is saliva. There exist systems for sampling saliva in order for example to carry out genetic analyses. It has been demonstrated that a sample of saliva can also be sufficient to detect a pathogenic agent, such as SARS-CoV-2, during a diagnostic test.

However, a problem that arises is controlling the quantity of sample collected. In order to be able to effect a diagnostic test on the sample of liquid, and in particular of saliva, the quantity of sample collected must be compatible with the predefined volume of inactivation liquid that will be mixed with the sample in order to assure the transport and the analysis of the sample in complete safety. The collected quantity of sample, for example of saliva, must be sufficient to assure detection of the pathogenic agent during the diagnostic test but must not be too great to assure the inactivation of the pathogenic agent by a predefined volume of inactivation liquid. Existing systems for sampling saliva generally employ graduated funnels or funnels fixed to graduated tubes, but do not make it possible to prevent excessive filling of the tube by the sample donor, which can impede the carrying out of a reliable diagnostic test and/or give rise to risks when transporting or transferring the sample.

The present invention therefore aims to address at least partially one or more of the disadvantages mentioned hereinabove. In particular, the objective of the invention is to provide a container system to receive a sample of liquid that enables relatively easy and safe sampling whilst enabling control of the quantity of sample.

### SUMMARY OF THE INVENTION

To this end a first aspect of the present invention is directed to a container system for receiving a liquid sample characterized by the elements cited in claim 1. In particular, the container system for receiving a liquid sample comprises a receptacle that includes a substantially tubular part adapted to receive a predefined volume of the sample. This substantially tubular part has an open first end and an at least partly openable second end. The receptacle also includes a chamber at least partly surrounding the substantially tubular part, the chamber having an at least partially open upper part and a closed bottom. In an inventive manner the substantially tubular part passes through the bottom or extends through the closed bottom of the chamber so that the first end of the tubular part is raised to a distance from the closed bottom of the chamber, for example to a distance between approximately 5 mm and approximately 100 mm, preferably between approximately 10 mm and approximately 30 mm. Accordingly, the receptacle is able to receive a liquid sample in a relatively easy manner via the open first end of the substantially tubular part in a quantity predefined by the volume of said sensibly tubular part, the liquid exceeding that volume flowing from the first end of the substantially tubular part to the chamber around said substantially tubular part that serves as an overflow for the predefined volume. As the second end of the substantially tubular part is openable, the liquid sample can be collected in another container if necessary, for example in a laboratory beaker or vial in order to effect potential treatment thereof.

A sample of liquid is for example saliva or gargle, but may extend to any type of sample where it is required to pour a defined volume into a container. Biological samples such as urine, blood, plasma are also targeted, as well as samples such as water. A granular sample that has the same flow properties as liquids is also considered to be included.

The second end of the tubular part may preferably extend to a distance from the closed bottom so that the closed bottom of the chamber is located between the first end and the second end of the tubular part. In this way the evacuation of the liquid sample via the second end of the substantially tubular part is facilitated. Alternatively, the second end of the substantially tubular part could be located inside the chamber around the substantially tubular part.

The substantially tubular part may advantageously include a bottom at least partially sloping close to the second end. A slope of this kind may for example terminate at the second end and thus favor the flow of the liquid sample, in particular in the case of a viscous liquid, toward that second end. The at least partially sloping bottom may for example be produced as a toboggan run or otherwise.

The second end of the substantially tubular part may preferably comprise an orifice that preferably extends over at most one-half of a cross-section of the substantially tubular part, in particular at most one-third of a cross section of the substantially tubular part. This orifice is configured to enable evacuation of the liquid sample located in the volume defined by the substantially tubular part of the receptacle without any of the contents of the chamber of the receptacle being able to escape.

Alternatively, the at least partially openable second end of the substantially tubular part may comprise a unidirectional valve. A valve of this kind, also known as a check valve, can enable flow of fluid in one direction and prevent flow of fluid in the opposite direction. In this case a unidirectional valve may for example be openable by pressure. The valve may be adapted to retain a predefined volume of liquid sample when the pressure in said volume is below a predefined threshold and to allow a flow of the received liquid sample when said predefined pressure threshold is exceeded.

The upper part of the chamber may preferably extend beyond the first end of the substantially tubular part, which can increase the volume of the chamber serving as an overflow. A relatively large chamber can therefore facilitate use of the receptacle, in particular when the sample is a gargle. In this case the receptacle may advantageously include a connection chute that substantially radially connects at least a part of the open upper part of the chamber to the first end of the substantially tubular part. This connection chute may for example resemble a toboggan run and may be configured to direct the liquid sample toward the first end of the substantially tubular part. The receptacle, in particular the upper edge of the chamber, may preferably include a mouthpiece that is preferably connected to this connection chute. Said mouthpiece may more or less have the width of a human mouth. The connection chute may for example be reduced in size toward the first end of the substantially tubular part of the receptacle. Alternatively, the first end of the substantially tubular part may extend beyond the upper part of the chamber. Alternatively, if the first end of the substantially tubular part rises beyond the upper part of the chamber the first end of the substantially tubular part may include a mouthpiece.

The chamber of the receptacle may preferably be connected to the substantially tubular part of the receptacle in a removable manner. The chamber may for example be screwed or otherwise fixed onto the substantially tubular part. In this way the overflow may be separated from the container system relatively easily after use. Alternatively, the receptacle is formed in one piece.

The open first end of the substantially tubular part of the receptacle may advantageously be configured to be connected to a container, for example a cartridge or capsule, containing a liquid, for example an inactivation liquid or any other type of liquid, the container preferably being closed by a film that can be pierced or detached. The connection may for example be effected by screwing or otherwise. The open first end of the substantially tubular part may for example include a piercing element intended to pierce a film of the container when the container is connected to the receptacle. By causing this liquid to pass through the tubular part of the receptacle an effect of rinsing this tubular part is obtained, which is all the more advantageous when the liquid sample collected in the receptacle is relatively viscous and flows with some difficulty, as for example in the case of a sample of saliva.

The container system may preferably include an adaptor having a first end intended to be connected, preferably in separable manner, to the receptacle, in particular to the second end of the substantially tubular part. Said first end of the adaptor may for example be configured for direct connection, connection or coupling by screwing and include an interior groove corresponding to an exterior thread at the second end of the substantially tubular part, or vice-versa. As the person skilled in the art knows, the connection may also be effected otherwise, for example by clipping, by clamping or otherwise. A second end of the adaptor may for example be configured to enable connection or coupling to an existing laboratory test tube, beaker or vial in order to allow evacuation of the liquid sample into a test, analysis, reaction or preservation container. Thus the adaptor of the container system according to the invention enables use of the system in combination with existing test, preservation or analysis equipment.

The adaptor may advantageously include an orifice extending over at least part of a cross-section of the adaptor. This orifice is configured to enable evacuation of the liquid sample located in the receptacle, and to be more precise in the substantially tubular part, to another test, analysis, reaction or preservation container. The extent of said orifice is preferably adapted to the extent of the orifice of the second end of the substantially tubular part of the receptacle: the orifice of the adaptor may for example be larger than the orifice of the second end of the substantially tubular part of the receptacle to favor good flow of the liquid sample.

In one very favorable embodiment the connection between the adaptor and the receptacle may be mobile between a first position in which the orifice at the second end of the substantially tubular part and the orifice of the adaptor are aligned or coincide at least partly and a second position in which the orifice at the second end of the substantially tubular part and the orifice of the adaptor are offset and therefore do not intersect. In this second position the adaptor is able to provide a sealed closure of the orifice of the receptacle whereas in the first position the liquid sample can be evacuated from the receptacle. This mobility between said first position and said second position can be obtained for example by a screwing or unscrewing movement or otherwise as a function of the type of connection or coupling between the receptacle and the adaptor.

The adaptor may for example include an abutment adapted to halt the receptacle in the second position when the receptacle is connected to the adaptor. In this way the adaptor can be configured so as to provide a sealed closure of the receptacle when the receptacle is connected or coupled to the adaptor, thus providing a sample receiving position, and so as to enable the liquid sample to flow only at the time of at least partial disconnection or uncoupling of the receptacle from the adaptor.

The container system may preferably include a sample tube that has an open or openable first end adapted to be connected in a separable manner to the receptacle, preferably to the adaptor, in particular to the second end of the adaptor, and a second end comprising a sample storage chamber. This second end of the sample tube is closable or preferably closed. The sample storage chamber may extend over a part of or over substantially the entirety of the interior of the sample tube. This sample tube may be a vial or some other type of container, preferably a container adapted to be manipulated in a laboratory, for example in automated test procedures. The sample tube can therefore receive the liquid sample that has been collected by means of the receptacle and transferred into the sample tube, for example via the adaptor of the container system.

The second end of the substantially tubular part of the receptacle may preferably be adapted to be connected to the open or openable first end of the sample tube. In the case of a direct connection the system may dispense with an adaptor, which may facilitate the manipulation of the system by the user. The connection may for example be effected by screwing, clipping, clamping or any other known connection means. The second end of the substantially tubular part of the receptacle may for example include an internal screwthread which can be screwed onto an external screwthread on the sample tube. This screwthread, or any other connecting means, is preferably located downstream of any unidirectional valve or other type of evacuation orifice that forms the at least partly openable end of the substantially tubular part of the receptacle.

The sample storage chamber of the sample tube is preferably at least partly configured to be prefilled with a liquid, in particular an inactivation liquid. In this case the first end of the sample tube is preferably closed by a film that can be pierced or detached, by a disconnectable stopper or by any other openable closure means. The prefilled quantity of liquid may be chosen and adapted as a function of the volume of the substantially tubular part of the receptacle of the container system. The liquid may be any type of liquid for the stabilization, preservation, transformation, transportation, recovery, neutralization or inactivation of the sample or of a substance contained in the sample. Alternatively, the sample tube may be empty before collecting the collected liquid sample and the liquid, for example an inactivation liquid, may be supplied in some other way. Providing two liquids may also be envisaged, a first of which is prefilled in the sample tube and the other which can be supplied otherwise. This may also be the same liquid that is partly supplied in the sample tube and partly in some other way.

The container system may equally include at least one stopper able to provide substantially hermetically-sealed closure of the sample tube. The stopper may be adapted to be connected to the sample tube, preferably to the first end of the adaptor or preferably to the open first end of the substantially tubular part of the receptacle. The container system may therefore also include a stopper adapted to be connected directly to the open first end of the sample tube or to be connected to the sample tube via the adaptor, in particular via the first end of the adaptor, or via the adaptor and/or the receptacle, in particular via the open first end of the substantially tubular part of the receptacle. The container system may therefore for example also include two stoppers, a first of which is intended to be connected to the first end of the adaptor or to the open first end of the substantially tubular part of the receptacle and a second stopper, preferably a substantially hermetically-sealed stopper, intended to be connected directly to the open or openable first end of the sample tube after disconnecting the adaptor from the sample tube. The substantially hermetically-sealed stopper intended to close the sample tube after depositing the sample may if necessary be the same as or different from a stopper initially present on the sample tube. After disconnecting the receptacle from the adaptor or after disconnecting the chamber of the receptacle from the substantially tubular part of the receptacle, and after the liquid sample has been evacuated into the sample tube connected to the second end of the adaptor or to the second end of the substantially tubular part of the receptacle or into another container, the stopper can close the container system in sealed manner in order to enable safe transportation to a laboratory of the sample tube containing the liquid sample to be tested.

The stopper may preferably include a chamber intended to contain a liquid, the chamber being closed by a film that can be pierced or detached. Accordingly, the liquid, for example an inactivation liquid, can be evacuated from the stopper and be mixed with the liquid sample that is located in the sample tube or the container. The volume of the chamber of the stopper and therefore the quantity of liquid may be chosen and adapted as a function of the volume of the substantially tubular part of the receptacle of the container system. The liquid can be any type of liquid for the stabilization, preservation, transformation, transportation, recovery, or inactivation of the sample or of the substance contained in the sample. The film may be pierced manually. Alternatively the adaptor or the substantially tubular part of the receptacle may preferably include a piercing element intended to pierce the film of the stopper when the stopper is connected to the adaptor or respectively to the substantially tubular part of the receptacle, which can facilitate and render safe the manipulation of the container system. As mentioned above, the liquid, for example an inactivation liquid, can also be supplied in the sample tube or be supplied partly in the sample tube and partly in the stopper.

The stopper may preferably be adapted to be inserted at least partly into the open first end of the substantially tubular part of the receptacle. The stopper is thus able to provide a closure not only by its shape but also by force. In particular, by increasing the areas of contact between the stopper and the open first end of the substantially tubular part of the receptacle and therefore increasing the friction forces between these surfaces in contact, the stopper is able to provide substantially hermetically-sealed closure of the container system so that the system can be transported to a laboratory and manipulated under relatively safe conditions.

The stopper may advantageously be adapted upon insertion of the stopper into the open first end of the substantially tubular part of the receptacle to bring about a flow of the predefined volume of the liquid sample received via the at least partly openable second end of the substantially tubular part of the receptacle. The stopper can therefore exercise a dual function: in addition to providing the container system with a substantially hermetically-sealed closure, the stopper can increase the pressure in the volume that has received the liquid sample and thus push said sample, like a piston, through the second end of the substantially tubular part of the receptacle, for example through a unidirectional valve or via any other evacuation orifice, toward a sample tube connected to the second end of the substantially tubular part of the receptacle or to the second end of the adaptor or to any other container. Thus the manipulation of the system can be facilitated.

The stopper may preferably include a retaining element configured to retain the adaptor so as to enable simultaneous uncoupling of the stopper and the adaptor of the sample tube, which can facilitate and accelerate manipulation by a user or in an automated process, for example in a laboratory. This uncoupling of the stopper and of the adaptor can be done manually or by a machine, for example by unscrewing or otherwise depending on the type of connection used. If the stopper is adapted to be connected to the open first end of the substantially tubular part of the receptacle the second end of the substantially tubular part of the receptacle may preferably include a retaining element configured to retain the receptacle so as to enable simultaneous uncoupling of the stopper, the receptacle and the adaptor from the sample tube, thus providing the same advantage mentioned hereinabove.

A second aspect of the invention is a method of taking a sample of liquid characterized by the elements cited in claim 12. This method can produce one or more of the advantages cited hereinabove.

### BRIEF DESCRIPTION OF THE DRAWINGS

A preferred embodiment of the invention will be described with reference to the appended drawings in which

FIGS. 1A and 1B represent a front view and a side view respectively of a container system according to a preferred embodiment of the present invention;

FIGS. 2A and 2B represent a perspective view of the container system from FIGS. 1A and 1B during and after taking a liquid sample;

FIG. 3 represents a perspective view of the stopper of the container system from FIG. 1A;

FIGS. 4A and 4B represent views in section of the receptacle and of the adaptor of the container system from FIG. 2A;

FIG. 5 represents a perspective view of the adaptor of the container system from FIG. 1A;

FIG. 6 represents a view in section of a receptacle of a container system according to a second preferred embodiment of the present invention;

FIGS. 7A, 7B, 7C, 7D and 7E represent views in section of the container system according to this second embodiment;

FIGS. 8A, 8B and 8C represent schematic views in section of a container system according to an alternative embodiment.

### DETAILED DESCRIPTION OF THE INVENTION

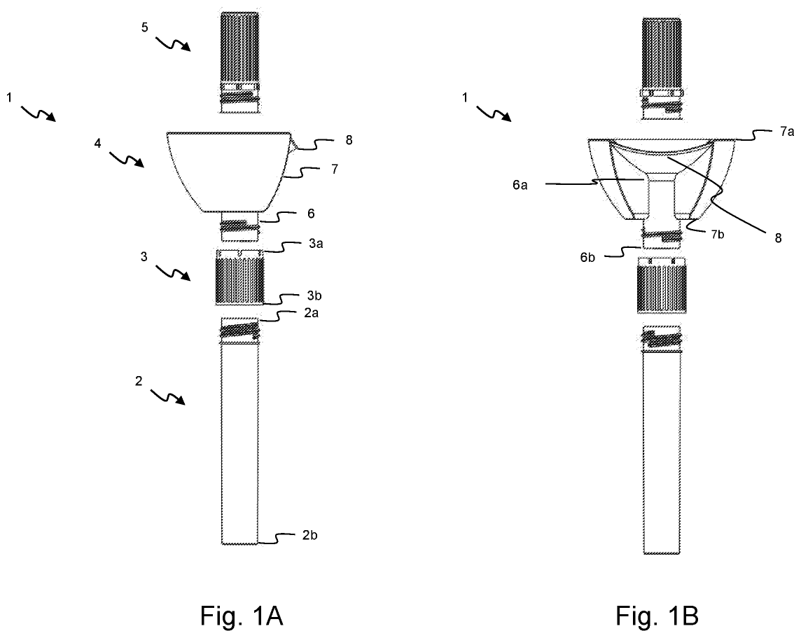

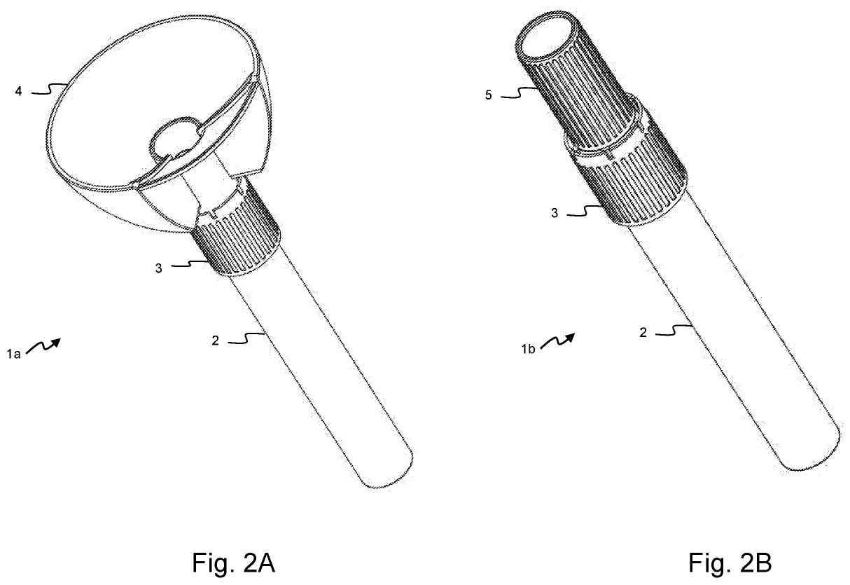

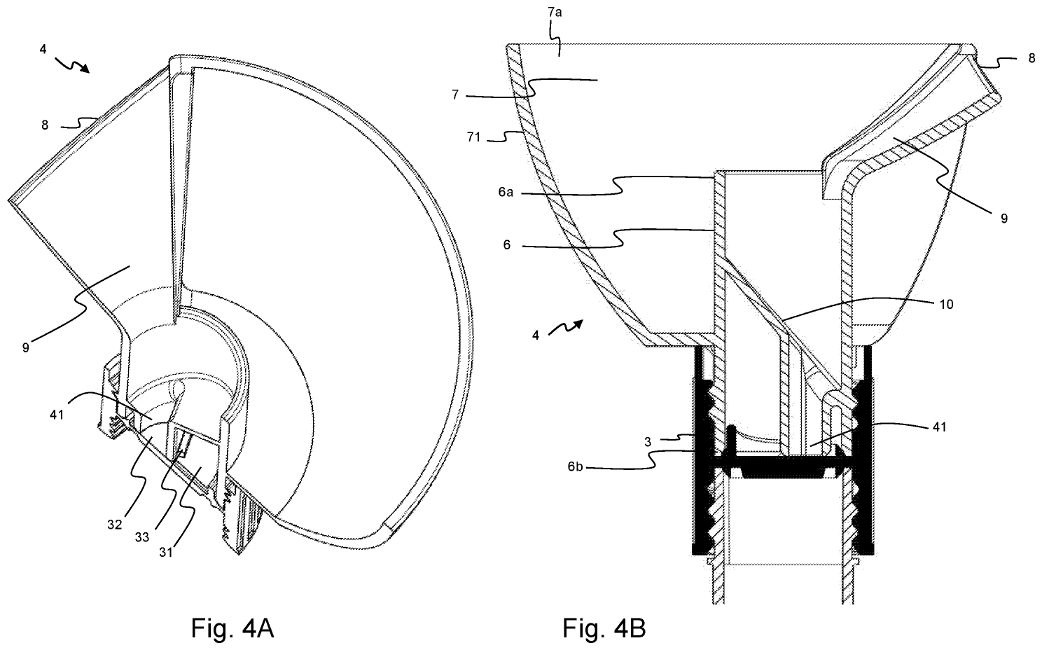

FIGS. 1A and 1B represent a front view and a side view respectively of a container system 1 according to a preferred embodiment of the present invention. This container system 1 may comprise four separate parts: a sample tube 2, an adaptor 3, a receptable 4 and a stopper 5. The sample tube 2 may be a standard laboratory sample tube, a test tube, a beaker or a sample tube dedicated to the container system 1. A total height of the sample tube 2 may be between approximately 60 mm and approximately 130 mm inclusive, preferably between approximately 70 mm and approximately 90 mm inclusive, for example approximately 80 mm. The interior volume of the tube is preferably between 2 ml and 10 ml. The sample tube 2 has an open first end 2a that provides access to a sample storage chamber inside the sample tube 2. The sample tube 2 of the container system 1 may be empty or may be partly pre-filled, for example with an inactivation liquid, or a transportation liquid or any other type of liquid. In this case the first end 2a may be provided with an openable closure element, for example a film that can be pierced or detached, or a stopper that can be detached, for example by unscrewing it. The second end 2b is preferably substantially conical on the interior, which is standard for certain sample tubes intended for diagnostic tests, but may for example have a substantially cylindrical or flat shape on the outside. The first end 2a may be provided with an exterior screwthread to enable easy screwing to the adaptor 3, in particular to the second end 3b of the adaptor 3. Other detachable or separable fixing means are possible. The first end 3a of the adaptor is configured to receive either the receptacle 4 or the stopper 5. In this embodiment the stopper 5 is not fixed to the receptacle 4 but to the adaptor 3, as illustrated in FIG. 2B. In other embodiments the stopper 5 may be configured to be fixed to the sample tube 2 directly or to the receptacle 4. The sample tube 2, the adaptor 3 and the stopper 5 preferably have a substantially cylindrical shape, although other shapes may be envisaged. The receptacle 4 comprises a substantially tubular part 6 and a chamber 7 at least partly surrounding this substantially tubular part 6. This chamber 7 comprises for example a flared part that may be interrupted by a mouthpiece 8. The chamber 7 has an at least partly open upper end 7a that is preferably entirely open. The substantially tubular part 6 has an open first end 6a and an at least partly openable second end 6b. The substantially tubular part 6 passes through or extends through the closed bottom 7b of the chamber 7 so that the first end 6a of the tubular part 6 rises to a distance from the closed bottom 7b of the chamber 7. The second end 6b of the substantially tubular part 6 extends beyond the closed bottom 7b of the chamber 7 so that the closed bottom 7b of the chamber 7 is located between the first end 6a and the second end 6b of the tubular part 6. Although the receptacle may resemble a funnel it is not a funnel because the liquid that is received in the chamber 7 at least partly surrounding its substantially tubular part 6 cannot be evacuated, in contrast to the function of a funnel. Only the volume of the liquid sample received in the substantially tubular part 6 can be evacuated. The receptacle 4 will be described in more detail with reference to FIG. 4.

FIGS. 2A and 2B represent a perspective view of the container system from FIGS. 1A and 1B during and after taking a sample of liquid. A first step of a method of taking a sample using the container system according to this preferred embodiment consists in assembling the parts of the system. The parts are preferably made of plastic material and/or rubber. The plastic used may be PE, PP, PUR, PS, PET and any other plastic suitable for this use or any combination of two or more of these plastics. The materials are preferably configured to withstand a temperature greater than 85° C. for the purposes of disinfection. A first sampling assembly 1a comprises the receptacle 4 that is connected to a first end 3a of the adaptor 3 and the sample tube 2 that is connected directly to or fixed in a separable manner to the second end 3b of the adaptor 3. These connections are preferably made by screwing, but other preferably direct connections may also be envisaged. The user can then deposit the liquid sample in the receptacle 4. The liquid sample is preferably saliva or gargle, but other types of samples of biological or non-biological liquid, such as urine, blood, plasma, water, etc. may be collected in the receptacle 4. Even if the container system is particularly advantageous for taking a sample of saliva with a view to diagnosing the presence of a pathogenic agent, it may equally be used in any other application where a sample of predefined volume must be taken, such as for example the detection of drugs, medication or pollutants. In order to guide the liquid sample toward the substantially tubular part 6 of the receptacle 4, the receptacle 4 may comprise a connection chute 9, such as a toboggan run, which connects at least a part of the open upper part 7a of the chamber 7 to the first end 6a of the substantially tubular part 6. This connecting chute 9 may comprise a mouthpiece 8 at its upper end in order to facilitate depositing a sample of liquid such as saliva, expectorated liquid phlegm or a gargle. The substantially tubular part 6 is adapted to receive a predefined volume of the liquid sample that will depend on the test to be carried out on the sample. According to the protocols of tests for detecting the SARS-CoV-2 virus, a volume of sample between approximately 0.1 ml and approximately 5 ml is needed, preferably between approximately 1 ml and 2 ml. The substantially tubular part 6 may include visible graduations to give the user an indication of the volume collected. To prevent a too great deposit of liquid sample the receptacle 4 is configured to provide an overflow zone, i.e. the chamber 7 surrounding at least partly the substantially tubular part 6. This chamber has a closed bottom 7b that is configured to receive the volume of liquid that overflows from the first end 6a of the substantially tubular part 6 of the receptacle 4. The liquid collected in this chamber 7 will not enter the sample tube afterwards. In this way it is relatively easy to control the quantity of liquid sample to be provided. It suffices to fill the substantially tubular part 6 of the receptacle 4 until the liquid overflows and the desired volume of the liquid sample can therefore be supplied. The next step of the sample taking method consists in transferring the liquid sample collected in the receptacle 4 to the sample tube 2. The receptacle 4 and in particular the substantially tubular part 6 of the receptacle 4 and the adaptor 3 are designed so as to enable the liquid sample to flow at the moment of the at least partial disconnection of the receptacle 4 from the adaptor 3. The receptacle 4 may preferably be unscrewed from the adaptor 3. At the moment of the at least partial unscrewing of the receptacle the liquid sample passes through the adaptor 3 to the sample tube 2. The receptacle 4 may thus be thrown away afterwards or be washed and disinfected for possible further use. In a following step the stopper 5 may be connected to, preferably screwed onto, the first end 3a of the adaptor 3, thus obtaining a second assembly 1b of the container system. The stopper 5 is configured to provide a substantially sealed closure of the sample tube 2 when the stopper 5 is connected to the adaptor 3. Additionally, or alternatively, the container system 1 may include a substantially hermetically-sealed stopper that can be connected to the sample tube 2 directly without using the adaptor 3. This substantially hermetically-sealed stopper may be mounted on the sample tube 2 after disconnecting the adaptor 3 from the sample tube 2. This substantially hermetically-sealed stopper may optionally be provided already in a detachment manner on the sample tube 2, for example when the sample tube 2 is prefilled with a liquid. As the stopper 5 or any other substantially hermetically-sealed stopper can close the sample tube 2, it is desirable to shake this second assembly 1b of the container system. This second assembly 1b can now be transported in complete safety to a laboratory or test center where it is manipulated, for example in an automated manner, to carry out therein a treatment or a test, for example a diagnostic test able to detect a viral load on the liquid sample.

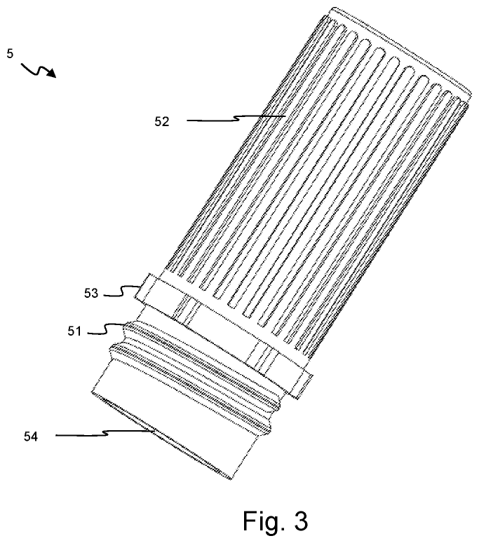

FIG. 3 represents a perspective view of the stopper of the container system from FIG. 1A. The lateral wall of the stopper 5 may include a screwthread 51 enabling screwing of the stopper 5 to the adaptor 3 and unscrewing it therefrom, or in another embodiment to the receptacle 4, in particular to the first end 6a of the substantially tubular part 6. Any other coupling or connecting mode can equally be envisaged. The lateral wall of the stopper 5 may also include longitudinal splines 52 adapted to facilitate the grip on the stopper 5 when screwing on or unscrewing the stopper 5. In order to facilitate and to accelerate manipulation of the container system manually or at the treatment or test center, uncoupling, preferably unscrewing, the stopper 5 can equally drive unscrewing of the adaptor 3. To this end the stopper 5 may include one or more retaining elements 53, for example sawtooth mouldings on a circumference of the lateral wall of the stopper 5, which are configured to grip corresponding elements of the adaptor 3 and to retain the adaptor 3 during unscrewing of the stopper 5. The stopper 5 may include a chamber intended to contain a liquid, the chamber being closed by a film 54 that can be pierced. The liquid may be any type of liquid suitable for the stabilization, preservation, transformation, transportation, recovery, neutralization or inactivation of the sample or of a substance contained in the sample. One example of an inactivation liquid is guanidium thiocyanate. Other liquids known to the person skilled in the art can equally be envisaged. The film 54 is configured to seal the stopper 5 when the stopper contains a liquid. This film is preferably a plastic or aluminum film or a film of a mixture of aluminum and plastic. The film may for example be hot-glued onto the stopper. The volume of inactivation liquid is substantially appropriate to the volume of the liquid sample. In the event of using the container system for a SARS-CoV-2 test, the stopper is preferably designed to be able to receive a volume of liquid between approximately 1 ml and approximately 3 ml, for example a volume of approximately 1.5 ml of the liquid, for example the inactivation liquid. After piercing the film, preferably during connection, for example during screwing of the stopper 5 onto the adaptor 3, or alternatively on connecting the stopper 5 to the first end 6a of the substantially tubular part 6, the inactivation liquid flows into the sample tube 2, which prevents contact of the user with the inactivation liquid. At the same time the flow of the liquid via the adaptor 3 or via the first end 6a of the substantially tubular part 6 of the receptacle 4 and the adaptor 3 enables rinsing of these parts, which is particularly advantageous in the case of a relatively viscous sample such as saliva. Providing two stoppers may equally be envisaged: one as described hereinabove, which may then be regarded as similar to a container containing a liquid, such as a cartridge or a capsule, and another, substantially hermetically-sealed stopper can be connected to the sample tube 2 directly or via the adaptor 3, and configured to seal the sample tube 2. According to an alternative embodiment the inactivation liquid may also be supplied in the sample tube 2 of the container system 1, which can be partly prefilled. After the step of transferring the collected liquid sample in the receptable 4 to the sample tube 2, the adaptor 3 may be disconnected from the sample tube 2 and discarded or disinfected. In a subsequent step a substantially hermetically-sealed stopper may be connected to, preferably screwed onto, the first end 2a of the sample tube 2. Combining these two methods may equally be envisaged by providing a sample tube prefilled with liquid and a stopper 5 containing a liquid. The sample tube 5 and the stopper 2 may each contain the same liquid or each may contain a different liquid.

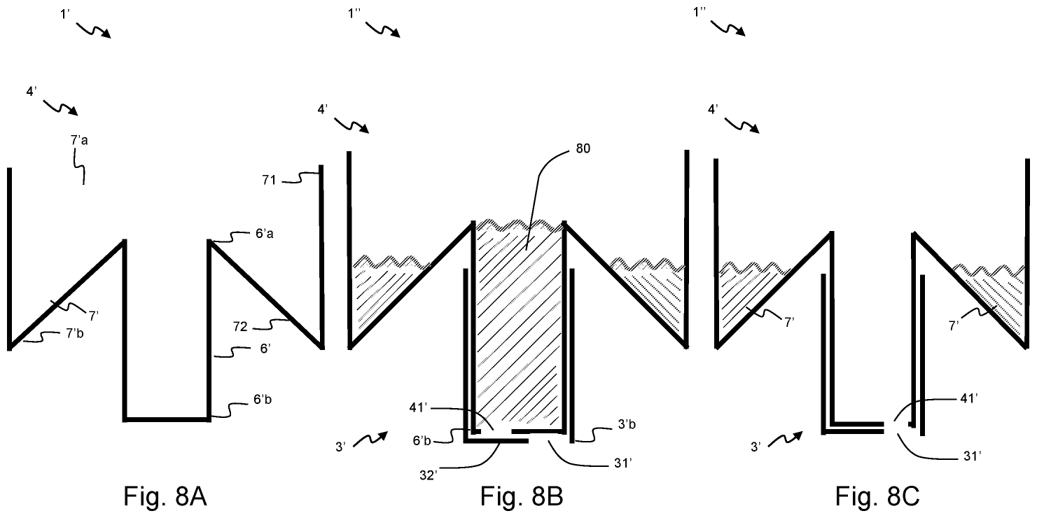

FIGS. 4A and 4B represent views in section of the receptacle and of the adaptor of the container system from FIG. 2A. The upper part 7a of the chamber 7 may rise beyond the first end 6a of the substantially tubular part 6 of the receptacle 4 but could also be lower. The lateral wall 71 of the chamber 7 may have a flared shape or an at least partly substantially conical shape, but a straight wall is equally possible as shown by the embodiment illustrated in FIG. 8A. As explained above, the receptacle 4 of this preferred embodiment comprises a connection chute 9 that connects in a substantially radial manner at least a part of the open upper part 7a of the chamber 7 to the first end 6a of the substantially tubular part 6 in order to guide the liquid sample toward to the opening of the substantially tubular part 6 of the receptacle 4. The substantially tubular part 6 preferably comprises corridors or bridges in order to favor the flow of the liquid sample, which may be relatively viscous, such as saliva. The substantially tubular part 6 comprises for example an at least partly sloping bottom 10 near the second end 6b to increase the area of contact and to favor the flow. The material will preferably be polished and/or varnished or a coating may be deposited on the material in order to favor the flow of the liquid sample. The receptacle 4 comprises an orifice 41 extending over at most a part of a cross-section of the adaptor 3. The orifice 41 preferably extends over at most one-third of the cross-section of the adaptor. The adaptor 3 equally comprises an orifice 31 extending over at most part of a cross-section of the adaptor 3, for example over approximately two-thirds of the cross-section of the adaptor 3. In this way the cross-section of the adaptor 3 also comprises a closed part 32 that extends for example over one-third of the adaptor 3 and that is able to close the receptacle 4 in sealed manner. The connection between the adaptor 3 and the receptacle 4 is preferably mobile between a first position in which the orifice 41 of the second end 6b of the substantially tubular part 6 and the orifice 31 of the adaptor 3 are at least partly aligned and a second position in which the orifice 41 of the second end 6b of the substantially tubular part 6 and the orifice 31 of the adaptor are offset, as illustrated in FIG. 4A. The first position therefore provides fluidic communication between the receptacle 4 and the adaptor 3, whereas the second position prevents any flow of the liquid sample out of the receptacle 4.

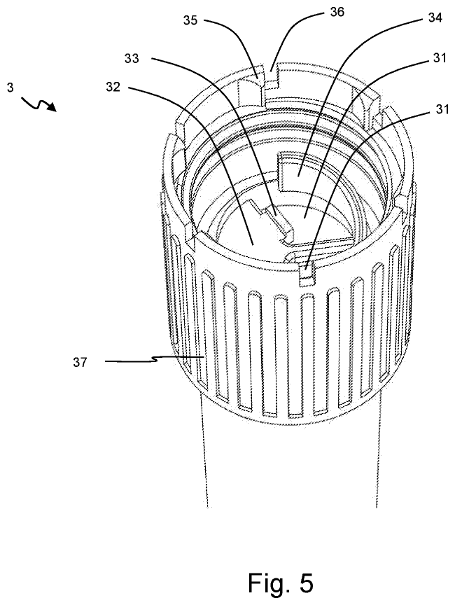

FIG. 5 represents a perspective view of the adaptor of the container system from FIG. 1A. The movement between the first position and the second position is for example a movement in rotation, for example an unscrewing movement. The adaptor 3 may for example include an abutment 33 adapted to halt the receptacle 4 in said second, closure position when the receptacle 4 is connected to the adaptor 3. Accordingly, after screwing the receptacle 4 onto the adaptor 3 the receptacle 4 is automatically located in the correct position to deposit a liquid sample. At the moment of disconnection, for example of unscrewing the receptacle 4 from the adaptor 3, the orifice 41 will go through a position aligned with the orifice 31 of the adaptor 3 that establishes fluidic communication between the receptacle 4 and the adaptor 3 and therefore enables flow of the liquid sample to another container, for example to the dedicated sample tube 2. The adaptor 3 may equally include a piercing element 34 intended to pierce the film 54 of the stopper 5 when the stopper 5 is connected to the adaptor 3. Alternatively, if the stopper 5 is configured to be connected to the open first end 6a of the tubular part 6 of the receptacle 4, the tubular part 6 of the receptacle 4 may be provided with a piercing element of this kind. This piercing element 34 may for example be a chimney or a circular arc wall with a cutting edge that extends at least partly radially. This piercing element 34 is preferably of relatively low height in order only to pierce the film 54 of the stopper 5 at the moment when the stopper 5 is already properly connected to the adaptor 3. The adaptor 3 may also include retaining elements 35 that correspond to retaining elements 53 of the stopper 5 and come to be gripped by said retaining elements 53 of the stopper 5 in order to enable simultaneous disconnection of the stopper 5 and the adaptor 3. The upper edge of the lateral wall of the adaptor 3 may include one or more slots 36 configured to allow a slight radial separation of the lateral wall when screwing the stopper 5 onto the adaptor 3, which facilitates relatively flexible screwing by allowing the retaining elements 53 of the stopper to pass over the retaining elements 35 of the adaptor 3. The asymmetric shape of these retaining elements 35 of the adaptor 3 enables the stopper 5 to be screwed onto the adaptor 3 but drives the unscrewing of the adaptor 3 when unscrewing the stopper 5. Like the stopper 5, the adaptor 3 may equally be provided with longitudinal grooves or splines 37 on the lateral exterior wall in order to improve the grip on the adaptor 3.

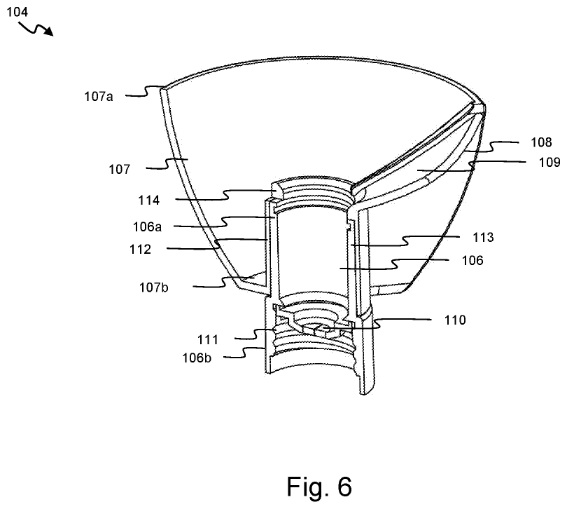

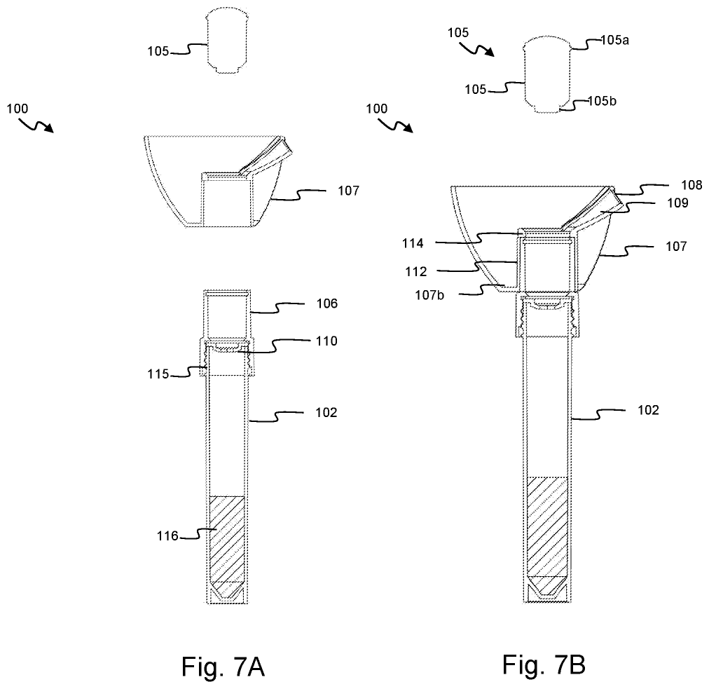

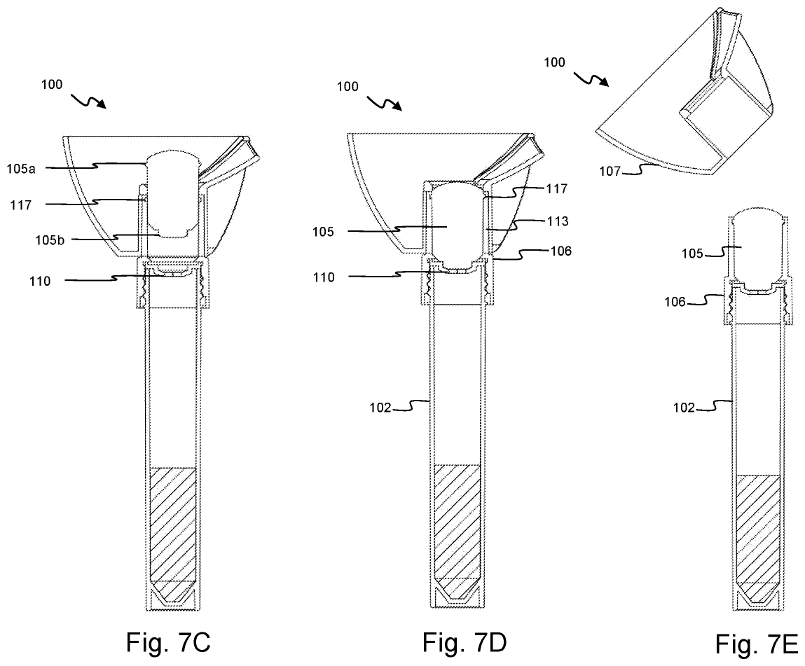

FIG. 6 represents a view in section of a receptacle 104 of a container system 100 according to a preferred second embodiment of the present invention. Just like the receptacle 4 from FIGS. 4A and 4B, the receptacle 104 comprises a substantially tubular part 106 and a chamber 107 at least partly surrounding that substantially tubular part 106. This chamber 107 comprises for example a flared part that may be interrupted by a mouthpiece 108. The chamber 107 has an at least partly open upper part 107a that is preferably entirely open. The substantially tubular part 106 has an open first end 106a and an at least partly openable second end 106b. The substantially tubular part 106 passes through or extends through the closed bottom 107b of the chamber 107 so that the first end 106a of the tubular part 106 rises to a distance from the closed bottom 107b of the chamber 107. The second end 106b of the substantially tubular part 106 extends beyond the closed bottom 107b of the chamber 107 so that the closed bottom 107b of the chamber 107 is located between the first end 106a and the second end 106b of the tubular part 106. The receptacle 104 equally comprises a connection chute 109 which connects substantially radially at least part of the open upper part 107a of the chamber 107 to the first end 106a of the substantially tubular part 106 in order to guide the liquid sample toward the opening of the substantially tubular part 106 of the receptacle 104. Unlike the first embodiment, the at least partly openable second end 106b of the substantially tubular part 106 comprises a unidirectional valve 110. This valve 110 is configured to allow passage or flow of a liquid sample collected in the substantially tubular part 106 to another container, for example to a sample tube 102, as illustrated in FIGS. 7A to 7E. This valve 110 is also configured to prevent the return of liquid contained for example in a connected sample tube. The valve 110 may be located at a required height of the substantially tubular part 106 as a function of the required volume of the substantially tubular part 106. This embodiment of the receptacle 104 further differs from the first embodiment in that the second end 106b of the substantially tubular part 106 is configured so as to be connected directly to the sample tube 102, in particular to the open or openable first end 102a of the sample tube 102. To this end the second end 106b of the substantially tubular part 106 may include an internal screwthread 111 that can be screwed onto an external screwthread on the sample tube 102. This screwthread or any other connecting means is preferably located downstream of a unidirectional valve 110 or some other type of evacuation orifice that forms the at least partially openable second end 106b of the substantially tubular part 106 of the receptable 104. A third difference from the first embodiment is that the chamber 107 can be connected to the substantially tubular part 106 in a separable manner. This connection may be made in various ways, for example by screwing, clipping, clamping or any other known connecting means. The chamber 107 may for example include a substantially central and substantially tubular wall 112 that tightly surrounds a lateral wall 113 of the substantially tubular part 106. An upper edge 112a of this wall 112 may extend radially inward in order to constitute a stop or abutment 114 for the wall 113 of the substantially tubular part 106 and the chamber 107 is mounted on the substantially tubular part 106.

FIGS. 7A, 7B, 7C, 7D and 7E represent views in section of the container system 100 according to this second embodiment. The container system 100 may include a sample tube 102, a stopper 105 and a receptacle 104 including two separable parts: the chamber 107 and the substantially tubular part 106 shown separated in FIG. 7A. The sample tube 102 in itself does not differ from the sample tube 2 of the first embodiment. The first end 102a may be provided with an external screwthread 115 to enable easy screwing on of the receptacle 104, in particular screwing it onto the second end 106b of the substantially tubular part 106 of the receptacle 104. Other detachable or separable fixing means are possible. The sample tube 102 of the container system 100 may be empty or preferably partly prefilled, for example with an inactivation liquid 116 or a transport liquid or any other type of liquid, for example with a quantity of approximately 1.5 ml of inactivation liquid or more or less as required. In order to prevent any contact between a user and this inactivation liquid 116 the sample tube 102 may be provided with an openable closure. In one advantageous embodiment the sample tube 102 prefilled with an inactivation liquid 116 may be supplied to the user with the substantially tubular part 106 of the receptacle 104 including a unidirectional valve 110 connected to, for example screwed onto, the sample tube 102. The unidirectional valve 110 can then provide a substantially sealed closure of the sample tube 102. It is obvious to the person skilled in the art that other closure means of the sample tube 102 may be envisaged. A user can then connect the chamber 107 of the receptacle 104 to the substantially tubular part 106 of the receptacle 104, for example for sliding the substantially central wall 112 of the chamber 107 on the substantially tubular part 106 until the first end 106a of the substantially tubular part 106 comes into contact with the abutment 114. This connection may also be made differently, for example by screwing, clipping or otherwise.

As shown in FIG. 7B, the container system 100 is now ready to receive a liquid sample. The user can for example supply or deposit this sample by placing the mouthpiece 108 in their mouth and spitting saliva or gargle for example into the receptacle 104. The liquid will flow along the connection chute 109 toward the substantially tubular part 106, which will collect a predefined volume of it. Surplus from the sample of the liquid will flow over the first end 106a of the substantially tubular part 106 and will be collected in the chamber 107, in particular at the closed bottom 107b of that chamber 107 surrounding the substantially tubular part 106 of the receptacle 104.

In a subsequent step the liquid sample received in the substantially tubular part 106 must be able to flow toward another container, preferably into the sample tube 102 that is connected to the receptacle 104, in order for it to be able to be examined in a laboratory. To this end the stopper 105 is configured to be inserted in the substantially tubular part 106 of the receptacle 104, as FIG. 7C shows. In a highly advantageous manner the diameter of the stopper is chosen to provide a substantially sealed closure of the substantially tubular part 106 of the receptacle 104. Accordingly, inserting the stopper 105 in the substantially tubular portion 106 can increase the pressure in said substantially tubular part 106, like a piston. This increase in pressure can then cause opening of the unidirectional valve 110 and cause flow of the predefined volume of the liquid sample received via the at least partly openable second end 106b of the substantially tubular part 106 of the receptacle 104, in particular via this unidirectional valve 110. FIGS. 7C, 7D and 7E show neither the liquid sample nor the unidirectional valve in the open position, but the person skilled in the art will understand the principle. The unidirectional valve 110 may be made of a relatively flexible material, for example consisting of an elastomer, preferably of silicone, or of rubber, or any other material of elastomer type. The valve may have a somewhat pointed V shape or a flattened U shape or any other appropriate shape. The valve may include a single slot or a plurality of slots, for example two slots arranged in the form of a cross. The unidirectional valve 110 may be attached, for example wedged or glued or otherwise mounted, inside the interior wall 113 of the substantially tubular part 106 of the receptacle.

Apart from being able to cause flow of the liquid sample the stopper 105 may equally be configured, in conjunction with the substantially tubular part 106 of the receptacle 104, to provide a substantially sealed closure of the sample tube 102. The stopper 105, in particular a first end of the stopper 105, may for example include a flange 105a extending radially outward. The substantially tubular part 106, in particular a first end 106a, in particular the interior of the lateral wall 113, may include a corresponding groove 117 adapted to receive said flange 105a of the stopper 105. This flange 105a can assist in arriving at a correct location of the stopper 105 by preventing excessive depression of the stopper 105 into the substantially tubular part 106. Said flange 105a may at the same time improve the seal of the sample tube 102. The stopper 105, in particular the second end 105b of the stopper 105, may have a shape corresponding to the shape of the unidirectional valve 110. Accordingly, after correct placing of the stopper 105, the second end 105b of the stopper 105 can espouse the shape of the unidirectional valve 110 and thus contribute to the substantially sealed closure of the sample tube 102, as shown in FIG. 7D.

Finally, as FIG. 7E shows, the chamber 107 of the receptacle 104 may be separated from the substantially tubular part 106 in the same manner as on assembly, for example by sliding, turning, unscrewing or any other means of disconnection. Accordingly, surplus liquid sample collected in the chamber 107 may be discarded. Alternatively, the chamber 107 could form a single part with the substantially tubular part 106 of the receptacle 104 or could be inseparably fixed above it. The sample tube 102 may be closed in substantially sealed manner by the stopper 105 inserted in the substantially tubular part 106 of the receptacle the second part 106b of which remains connected to, for example screwed onto, the open first part 102a of the sample tube 102. The sample tube can thus be sent to a laboratory safely. In the laboratory the substantially tubular part 106 of the receptacle 104 including the unidirectional valve 110 may be disconnected, for example unscrewed, for example in an automated manner, from the sample tube 102 without this necessitating any manipulation of the stopper 105 and the liquid sample can undergo the required tests and/or analyses.

FIGS. 8A, 8B and 8C represent schematic views in section of a container system according to an alternative embodiment. In FIG. 8A the container system 1′ comprises only a receptacle 4′ that may possibly be used in combination with a sample tube, a laboratory vial, a test tube or any other existing container. As is the case in the preferred embodiment the receptacle 4′ includes a substantially tubular part 6′ adapted to receive a predefined volume of the liquid sample. The substantially tubular part 6′ has an open first end 6′a and an at least partly openable second end 6′b. The opening of the second end 6′b may be provided for example by an orifice covered by a film or by a bottom provided with a stopper or a valve or by any other means known to the person skilled in the art. This second end may for example have a substantially cylindrical shape as illustrated or alternatively a substantially conical shape in order to improve the flow of the liquid sample. The receptacle 4′ equally includes a chamber 7′ at least partly surrounding the substantially tubular part 6′, the chamber 7′ having an at least partly open or completely open upper part 7′a and a closed bottom 7′b. The substantially tubular part 6′ extends through the closed bottom part 7′b of the chamber 7 so that the first end 6′a of the substantially tubular part 6′ rises to a distance from the closed bottom 7′b of the chamber 7′, in this case from the lowest point of the closed bottom 7′b. The first end 6′a may for example rise to a distance of between approximately 5 mm and approximately 100 mm from the closed bottom 7′b of the chamber 7′. In this embodiment the lateral wall 71 of the chamber 7′ is a substantially straight wall and does not form a flared portion as in a funnel. The bottom 7′b of the chamber 7′ is formed by an inclined part 72 surrounding the substantially tubular part 6′ which joins the lateral wall 71 at a height between the first end 6′a and the second end 6′b of the substantially tubular part 6′ in order to form an overflow around the substantially tubular part 6′. The inclined part 72 is configured to direct the liquid that overflows from the substantially tubular part 6′ toward the bottom 7′b of the chamber 7 so that the substantially tubular part 6′ contains the desired volume of liquid sample.

In FIGS. 8B and 8C the container system 1″ comprises a receptacle 4′ and an adaptor 3′. As in the preceding embodiment illustrated in FIG. 8A the container system may be used in combination with a sample tube or a laboratory vial or any other existing container. The receptacle 4′ is similar to the embodiment from FIG. 8A as well. The difference is in the functioning and the interaction between the receptacle 4′ and the adaptor 3′. As FIG. 8B shows, a section of the adaptor 3′ shows in section a substantially U shape having a first end 3′a configured to be connected to the receptacle, and in particular to the second end 6′b of the substantially tubular part 6′ of the receptacle 4′. The adaptor 3′, in particular a second end 3′b of the adaptor 3′, includes an orifice 31′ extending over at most part of a cross-section 30 of the adaptor 3′. As the orifice extends over only part of the cross-section there remains a closed part 32′ of the cross-section 30. This closed part 32′ comes to provide sealed closure of the receptacle 4′ of which the second end 6′b of the substantially tubular part 6 also includes an orifice 41′ that equally extends over at most half of a cross-section of a substantially tubular part 6′. The connection between the adaptor 3′ and the receptacle 4′ is preferably mobile between a first position illustrated in FIG. 8C in which the orifice 41′ of the second end of the substantially tubular part 6′ and the orifice 31 of the adaptor 3′ are at least partly aligned and a second position illustrated in FIG. 8B in which the orifice 41′ of the second end of the substantially tubular part 6′ and the orifice 31′ of the adaptor are offset and therefore not aligned. This second position results in a closed position of the receptacle 4′ whereas the first position results in an open position, for example in the presence of a container in order to decant the predefined volume of the liquid sample 80 from the receptacle to a laboratory container without the liquid present in the chamber 7′ flowing.

Although the present invention has been illustrated with reference to specific embodiments, the person skilled in the art will understand that the invention is not limited to the details of the illustrative embodiments and that the present invention may be reduced to practise with numerous modifications without departing from the scope of the invention. The embodiments must be considered as illustrative and not restrictive, the scope of the invention being defined by the claims hereinafter rather than by the foregoing description. Any modification entering into the meaning or the equivalence of the claims is intended to be included. In other words, it is intended to cover all modifications, variations or equivalences that fall within the scope of the underlying basic principles and the essential features of which are claimed in this patent application. The reader of this patent application will understand that the words “comprising” and “including” do not exclude other elements or steps and that the words “a” or “an” do not exclude a plurality. The reference signs in the claims must not be considered as limiting the claim concerned. The terms “first”, “second”, “third”, “a”, “b”, “c”, etc. are introduced to distinguish different elements or steps and do not necessarily describe a sequential or chronological order. Likewise, the terms “upper”, “lower”, “above”, “below”, etc. are introduced for descriptive purposes and not necessarily to designate relative positions. It will be clear that these terms are interchangeable under appropriate conditions and that embodiments of the invention are capable of being operative in accordance with the present invention in other sequences or in orientations that differ from those described hereinabove or illustrated.

1.-12. (canceled)

13. A container system for receiving a liquid sample comprising a receptacle including:
a substantially tubular part adapted to receive a predefined volume of the sample, the substantially tubular part having an open first end and an at least partly openable second end;
a chamber at least partly surrounding the substantially tubular part, the chamber having an at least partly open upper part and a closed bottom;
the substantially tubular part passing through the closed bottom of the chamber so that the first end of the substantially tubular part rises to a distance from the closed bottom of the chamber.

14. The container system according to claim 13, in which the upper part of the chamber rises beyond the first end of the substantially tubular part.

15. The container system according to claim 14, in which the receptacle comprises a connection chute which connects at least part of the open upper part of the chamber to the first end of the substantially tubular part.

16. The container system according to claim 13, in which the chamber is connected to the substantially tubular part in a separable manner.

17. The container system according to claim 13, in which the at least partly openable second end of the substantially tubular part comprises a unidirectional valve.

18. The container system according to claim 13, including a sample tube having an open or openable first end adapted to be connected in separable manner to the receptacle and a second end comprising a sample storage chamber.

19. The container system according to claim 18, in which the second end of the substantially tubular part of the receptacle is adapted to be connected to the open or openable first end of the sample tube.

20. The container system according to claim 18, in which the sample storage chamber of the sample tube is configured to be at least partly prefilled with a liquid, in particular an inactivation liquid.

21. The container system according to claim 18, including at least one stopper adapted to provide substantially hermetically sealed closure of the sample tube.

22. A container system according to claim 21, in which the stopper can be inserted at least partly in the open first end of the substantially tubular part of the receptacle.

23. The container system according to claim 10, in which the stopper is adapted, upon insertion of the stopper in the open first end of the substantially tubular part of the receptacle, to cause the predefined volume of the liquid sample received via the at least partly openable second end of the substantially tubular part of the receptacle to flow.

24. A method of taking a liquid sample, including the steps of:
providing a container system according to claim 13;
optionally connecting the chamber of the receptacle to the substantially tubular part of the receptacle;
optionally connecting the receptacle to the sample tube, which is optionally at least partly prefilled with a liquid;
providing the liquid sample to the receptacle;
opening the second end of the substantially tubular part in order to sample the predefined volume of the liquid sample, the second end of the substantially tubular part preferably being opened by placing a stopper on the open first end of the substantially tubular part of the receptacle;
optionally disconnecting the chamber of the receptacle from the substantially tubular part of the receptacle.
